(19) United States
(12) Patent Application Publication
(10) Pub. No.: US 2004/0197268 A1
(43) Pub. Date: Oct. 7, 2004

(54) Method of imaging amyloid deposits using substituted rhodanine derivatives

(76) Inventors: Corinne Elizabeth Augelli-Szafran, Ann Arbor, MI (US); Shelly Ann Glase, Ann Arbor, MI (US); Terri Stoeber Purchase, Ann Arbor, MI (US)

Correspondence Address: OROAMERICA INC, LEGAL DEPARTMENT, 443 NORTH VARNEY STREET, BURBANK, CA, 91502

(21) Appl. No.: 10/829,128

(22) Filed: Apr. 21, 2004

Related U.S. Application Data
(63) Continuation of application No. 10/009,367, filed on Dec. 7, 2001.
Application No. 10/009,367 is a 371 of international application No. PCT/US00/15072, filed on May 31, 2000.
(60) Provisional application No. 60/138,545, filed on Jun. 10, 1999.

(51) Int. Cl. A61K 49/00; A61K 31/426

(52) U.S. Cl. 424/9.6; 514/369

(57) ABSTRACT

The present invention provides a method of treating Alzheimer's disease using a compound of Formula I

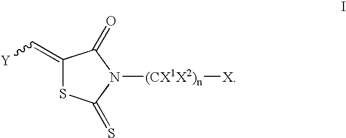

Also provided is a method of inhibiting the aggregation of amyloid proteins using a compound of Formula I and a method of imaging amyloid deposits using substituted rhodanine derivatives.

### FIELD OF THE INVENTION

This invention relates to a method of inhibiting amyloid protein aggregation and imaging amyloid deposits. More particularly, this invention relates to a method of inhibiting amyloid protein aggregation in order to treat amyloid aggregation disorders such as Alzheimer's disease using substituted rhodanine derivatives.

### BACKGROUND OF THE INVENTION

Amyloidosis is a condition characterized by the accumulation of various insoluble, fibrillar proteins in the tissues of a patient The fibrillar proteins that comprise the accumulations or deposits are called amyloid proteins. While the particular proteins or peptides found in the deposits vary, the presence of fibrillar morphology and a large amount of β-sheet secondary structure is common to many types of amyloids. An amyloid deposit is formed by the aggregation of amyloid proteins, followed by the further combination of aggregates and/or amyloid proteins.

The presence of amyloid deposits has been shown in various diseases, each with its particular associated protein, such as Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Alzheimer's disease, Down's syndrome, Scrapie, Creutzfeldt-Jacob disease, Kuru, Gerstmann-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, sickle cell anemia, Parkinson's disease, and Islets of Langerhans diabetes type 2 insulinoma.

Alzheimer's disease is a degenerative brain disorder characterized clinically by progressive loss of memory, cognition, reasoning, judgement, and emotional stability that gradually leads to mental deterioration and ultimately death. Because Alzheimer's disease and related degenerative brain disorders are a major medical issue for an increasingly aging population, the need for new treatments and methods for diagnosing the disorders are needed.

A simple, noninvasive method for detecting and quantitating amyloid deposits in a patient has been eagerly sought. Presently, detection of amyloid deposits involves histological analysis of biopsy or autopsy materials. Both methods have major drawbacks. For example, an autopsy can only be used for a postmortem diagnosis.

The direct imaging of amyloid deposits in vivo is difficult, as the deposits have many of the same physical properties (ie, density and water content) as normal tissues. Attempts to image amyloid deposits directly using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules has provided some selectivity on the periphery of tissues, but has provided for poor imaging of tissue interiors.

Thus, it would be useful to have a noninvasive technique for imaging and quantitating amyloid deposits in a patient. In addition, it would be useful to have compounds that inhibit the aggregation of amyloid proteins to form amyloid deposits. U.S. Pat. No. 5,523,314 describes certain rhodanines said to be useful for treating Alzheimer's disease. The present compounds differ in structure and are surprisingly potent inhibitors of amyloid aggregation.

### SUMMARY OF THE INVENTION

The present invention provides compounds having the Formula I:

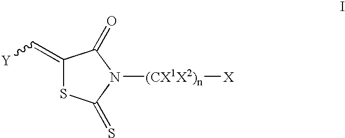

or a pharmaceutically acceptable salts thereof,

wherein:

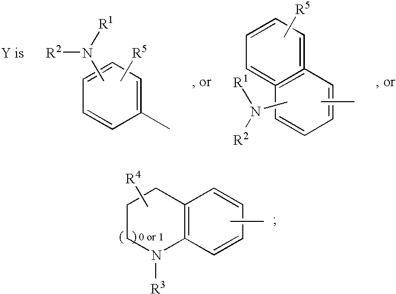

each n is independently 1 to 3 inclusive;

X¹ and X² are independently hydrogen or C₁-C₈ alkyl, or CH₂)_{y}-Z;

y is 0 to 4 inclusive;

Z is hydrogen, C₁-C₈ alkyl, C₃-C₈ cycloalkyl, C₁-C₈ perfluoroalkyl, C₂-C₈ alkenyl, phenyl, substituted phenyl, naphthyl, substituted naphthyl, —OH, —OC₁-C₈ alkyl, —SC₁-C₈ alkyl, —SO₃H, —CO₂H, —CO₂C₁-C₈ alkyl,

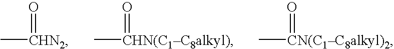

—NH₂, —NH(C₁-C₈alkyl), —N(C₁-C₈alkyl₂,

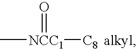

guanidinyl, thienyl, imidazolyl, thiazolyl, or indolyl;

R¹ and R² are independently C₁-C₈alkyl or CH₂)ₙ—C₃-C₆cycloalkyl, —(CH₂)ₙ-phenyl, or R¹ and R² taken together with the nitrogen atom to which they are attached form a cyclic structure selected from

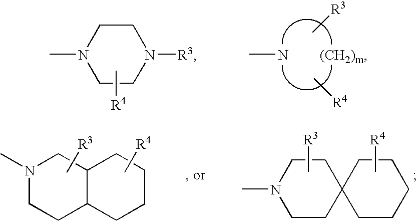

where R³ and R⁴ independently are hydrogen, C₁-C₈ alkyl, —(CH₂)ₙ-phenyl, or —(CH₂)ₙ cycloalkyl;

R⁵ is hydrogen, C₁-C₈ alkyl, halogen, or —CF₃;

each m is 2 to 8 inclusive;

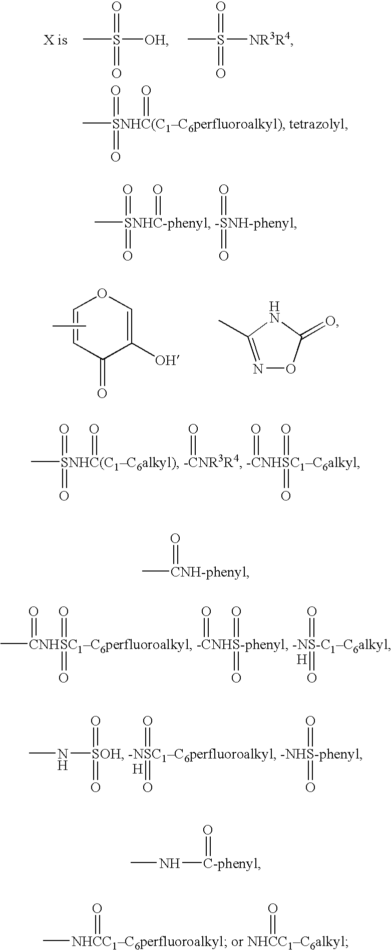

wherein phenyl includes substituted phenyl.

In a preferred embodiment of the compounds of Formula I, R¹ is methyl and R² is pentyl or hexyl.

Also preferred are compounds of Formula I wherein X¹ and X² both are hydrogen.

In another preferred embodiment of the compounds of Formula L

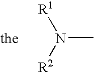

group is located at the para position on the aryl ring, for example 4-aminophenyl.

Also preferred are compounds of Formula I where Y has the Z geometry at the double bond.

In another preferred embodiment, R¹ and R² are taken together with the nitrogen to which they are attached to form a cyclic structure.

Also preferred are compounds wherein R² is —(CH₂)ₙ—C₃-C₆ cycloalkyl or —(CH₂)ₙ-phenyl when R¹ is C₁-C₈ alkyl.

Especially preferred compounds are benzylidenes of Formula II

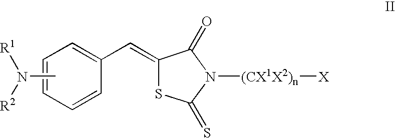

wherein n, X¹, X², and X are as defined above.

Further preferred compounds are naphthalenylmethylene derivatives of Formula III

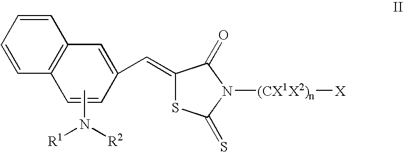

wherein R¹, R², X¹, X², n, and X are as defined above.

Still other preferred compounds are quinolinylmethylene derivatives of Formula IV

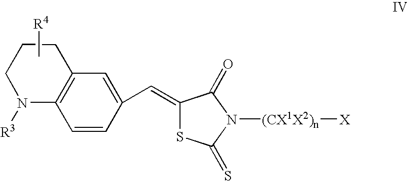

wherein R³, R⁴, X¹, X², n, and X are as defined above.

The most preferred invention compounds have Formula V

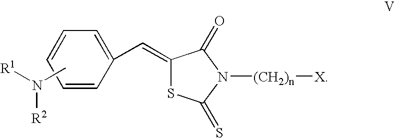

In a more preferred embodiment, the present invention provides the compounds:

(Z) 2-5{-[4{Hexyl-methyl-amino)-benzylidene]-4-oxo-2-thioxo-thiazolidin-3-yl}-ethanesulfonic acid;

(Z) 2-{5-[4-(Hexyl-methyl-amino)-benzylidene]4-oxo-2-thioxo-thiazolidin-3-yl}-ethanesulfonic acid methylamide;

(Z) 2-{5-[4-(Hexyl-methyl-amino)-benzylidene]4-oxo-2-thioxo-thiazolidin-3-yl}-ethanesulfonic acid trifluoroacetyl-amide;

(Z) 2-{5-[4-(Hexyl-methyl-amino)-benzylidene]4-oxo-2-thioxo-thiazolidin-3-yl}-N-methyl-acetaminde;

(Z) N-({5-[4-(Hexyl-methyl-amino)-benzylidene]4-oxo-2-thioxo-thiazolidin-3-yl}-acetyl)-methanesulfonamide;

(Z) N-{5-[4-(Dipentylamino-benzylidene)-4-oxo-2-thioxo-thiazolidin-3-yl]-acetyl}-methanesulfonamide;

(Z) C,C,C-Trifluoro-N-({5-[4-(hexyl-methyl-amino)-benzylidene]4-oxo 2-thioxo-thiazolidin-3-yl}-acetyl)-methanesulfonamide;

(Z) N-{5-[4-(Dipentylamino-benzylidene)₄-oxo-2-thioxo-thiazolidin-3-yl]-acetyl}-C,C,C-trifluoro-methanesulfonamide;

(Z) N-({5-[4(Hexyl-methyl-amino)-benzylidene]4-oxo-2-thioxo-thiazolidin-3-yl}-acetyl)benzenesulfonamide;

(Z) N-(2-{5-[4-(Hexyl-methyl-amino)-benzylidene]4-oxo-2-thioxo-thiazolin-3-yl}-ethyl)methanesulfonamide;

(Z) N-(2-{5-[4-(Hexyl-methyl-amino)-benzylidene]4-oxo-2-thioxo-thiazolidin-3-yl}-ethyl)-benzenesulfonamide;

(Z) C,C,C-Trifluoro-N-(2-{5-[4-(hexyl-methyl-amino)-benzylidene]J oxo-2-thioxo-thiazolidin-3-yl}-ethyl)-methanesulfonamide;

(Z) 2,2,2-Trifluoro-N-(2-(5-[4-(hexyl-methyl-amino)benzylidene]4-oxo-2-thioxo-thiazolidin-3-yl}-ethyl)-acetamide;

(Z) N-(2-{5-[4-Hexyl-methyl-amino)-benzylidene]4-oxo-2-thioxo-thiazolidin-3-yl}ethyl)-acetamide;

(Z) {5-[4-(Hexyl-methyl-amino)-benzylidene]4-oxo-2-thioxo-thiazolidin-3-yl}-methanesulfonic acid;

(Z) 5-[4-(Hexyl-methyl-amino)benzylidene]-3-(1H-tetrazol-5-ylmethyl)-2-thioxo-thiazolidin-4-one;

(Z) 5-(4-Dipentylamino-benzylidene)-3-(1H-tetrazol-5-ylmethyl)-2-thioxo-thiazolidin-4-one;

(Z) N-{[5-(4-Dibutylamino-benzylidene)-4-oxo-2-thioxo-thiazolidin-3-yl]-acetyl}-C,C,C-trifluoro-methanesulfonamide;

(Z) N-{[5-(4-Dibutylamino-benzylidene)-4-oxo-2-thioxo-thiazolidin-3-yl]-acetyl}-benzenesulfonamide;

(Z) 5-(4-Dibutylamino-benzylidene)-3-(1H-tetrazol-5-ylmethyl)-2-thioxo-thiazolidin-4-one;

(Z) N-{2-[5-(4-Dibutylamino-benzylidene)₄-oxo-2-thioxo-thiazolidin-3-yl]-acetyl}-methanesulfonamide;

(Z) N-{2-[54-Dipentylamino-benzylidene)-4-oxo-2-thioxo-thiazolidin-3-yl]-acetyl}-benzenesulfonamide;

(Z) 5-[(4aS,8aR)-4(Octahydro-isoquinolin-2-yl)-benzylidene]-3-(1H-tetrazol-5-ylmethyl)-2-thioxo-thiazolidin one;

(Z) N-(2-{5-[(4aS,8aR)₄-(Octahydro-isoquinolin-2-yl)-benzylidene]-4-oxo-2-thioxo-thiazolidin-3-yl}-acetyl)benzenesulfonamide;

(Z) N-{2-[5-4-Dibutylamino-benzylidene)-4-oxo-2-thioxo-thiazolidin-3-yl]-acetyl}-4-fluoro-benzenesulfonamide;

(Z) 2-[5-(4-Dibutylamino-benzylidene)-4-oxo-2-thioxo-thiazolidin-3-yl]-ethanesulfonic acid 4-fluoro-benzoylamide;

(Z) N-{2-[54-Dipentylamino-benzylidene)-4-oxo-2-thioxo-thiazolidin-3-yl]-acetyl}-4-fluoro-benzenesulfonamide;

(Z) 2-[5-(4-Dibutylamino-benzylidene)-4-oxo-2-thioxo-thiazolidin-3-yl]-ethanesulfonic acid benzoylamide;

(Z) 2-{5-[4-(Octahydro-isoquinolin-2-yl)-benzylidene]-4-oxo-2-thioxo-thiazolidin-3-yl}-ethanesulfonic acid benzoylamide;

(Z) 2-{5-[4-(Octahydro-isoquinolin-2-yl)-benzylidene]-4-oxo-2-thioxo-thiazolidin-3-yl}-ethanesulfonic acid 4-fluoro-benzoylamide;

(Z) 2-[5-(4-Dipentylamino-benzylidene)-4-oxo-2-thioxo-thiazolidin-3-yl]-ethanesulfonic acid 4-fluoro-benzoylamide;

(Z) 3-(5-Hydroxy-4-oxo-4H-pyran-2-ylmethyl)-5-[4-(octahydro-isoquinolin-2-yl)-benzylidene]-2-thioxo-thiazolidin-4-one;

(Z) 5-(4-Dibutylamino-benzylidene)-3-(5-hydroxy-4-oxo-4H-pyran-2-ylmethyl)-2-thioxo-thiazolidin-4-one;

(Z) 3-(5-Hydroxy-4-oxo-4H-pyran-2-ylmethyl)-5-[4-(4-propyl-piperidin-1-yl)-benzylidene]-2-thioxo-thiazolidin-4-one;

(Z) 5-[4[(4-Propyl-piperidin-1-yl)benzylidene]-3-(1H-tetrazol-5-ylmethyl)-2-thioxo-thiazolidin-4-one;

(Z) N-(2-{4-Oxo-5-[4-(4-propyl-piperidin-1-yl)-benzylidene]-2-thioxo-thiazolidin-3-yl}-acetyl)-benzenesulfonamide;

(Z) N-(2-{4-Oxo-5-[4-(4-propyl-piperidin-1-yl)-benzylidene]-2-thioxo-thiazolidin-3-yl}-acetyl)-methanesulfonamide;

(Z) 4-Fluoro-N-2-{5-[(4aS,8aR)-4-(octahydro-isoquinolin-2-yl)-benzylidene]-4-oxo-2-thioxo-thiazolidin-3-yl}-acetyl)-benzenesulfonamide;

(Z) 4-Fluoro-N-2-{4-oxo-5-[4-(4-propyl-piperidin-1-yl)-benzylidene]-2-thioxo-thiazolidin-3-yl}-acetyl)-benzenesulfonamide;

(Z) 2-[5-(4-Hexyl-methyl-amino-benzylidene)-4-oxo-2-thioxo-thiazolidin-3-yl]-ethanesulfonic acid 4-fluoro-benzoylamide;

(Z) N-({5-[4[(Octahydro-isoquinolin-2-yl)-benzylidene]4-oxo-2-thioxo-thiazolidin-3-yl}-acetyl)-methanesulfonamide;

(Z) N-({5-[4[(Octahydro-isoquinolin-2-yl)benzylidene]4-oxo-2-thioxo-thiazolidin-3-yl}-acetyl)-C,C,C-trifluoro-methanesulfonamide;

(Z) N-(2-{4-Oxo-5-[4-(4-propyl-piperidin-1-yl)-benzylidene]-2-thioxo-thiazolidin-3-yl}-acetyl)-C,C,C-trifluoro-methanesulfonamide;

(Z) 2-[5-(4-Dibutylamino-benzylidene)-4-oxo-2-thioxo-thiazolidin-3-yl]-ethanesulfonic acid methylamide;

(Z) 2-[5-(4-Dipentylamino-benzylidene)-4-oxo-2-thioxo-thiazolidin-3-yl]-ethanesulfonic acid methylamide;

(Z) 2-[5-(4-Hexyl-methyl-amino-benzylidene)₄-oxo-2-thioxo-thiazolidin-3-yl]-ethanesulfonic acid methylamide;

(Z) N-2-{4-Oxo-5-[4-(4-propyl-piperidin-1-yl)-benzylidene]-2-thioxo-thiazolidin-3-yl}-ethanesulfonic acid methylamide;

(Z) 2-{5-[4-(octahydro-isoquinolin-2-yl)-benzylidene]-4-oxo-2-thioxo-thiazolidin-3-yl}S-ethanesulfonic acid methylamide;

(Z) 2-{5-[4-(Octahydro-isoquinolin-2-yl)-benzylidene]-4-oxo-2-thioxo-thiazolidin-3-yl}S-ethanesulfonic acid trifluoroacetylamide;

(Z) N-2-{4-Oxo-5-[4-(4-propyl-piperidin-1-yl)-benzylidene]-2-thioxo-thiazolidin-3-yl}-ethanesulfonic acid trifluoroacetylamide;

(Z) 2-[5-(4-Dipentylamino-benzylidene4-oxo-2-thioxo-thiazolidin-3-yl]-ethanesulfonic acid trifluoroacetylaraide;

(Z) 2-[5-(4-Dibutylamino-benzylidene)-4-oxo-2-thioxo-thiazolidin-3-yl]-ethanesulfonic acid trifluoroacetylamide;

(Z) 2-[5-(4-Dipentylamino-benzylidene)-4-oxo-2-thioxo-thiazolidin-3-yl]-ethanesulfonic acid benzoylamide;

(Z) 2-[5-(4-Hexyl-methyl-benzylidene)-4-oxo-2-thioxo-thiazolidin-3-yl]-ethanesulfonic acid benzoylamide;

(Z) N-2-{4-Oxo-5-[4-(4-propyl-piperidin-1-yl)-benzylidene]-2-thioxo-thiazolidin-3-yl}-ethanesulfonic acid benzoylamide;

(Z) N-2-{4-Oxo-5-[4-(4-propyl-piperidin-1-yl)-benzylidene]-2-thioxo-thiazolidin-3-yl}-ethanesulfonic acid 4-fluoro-benzoylamide;

(Z) 2-[5-(4-Hexyl-methyl-benzylidene)-4-oxo-2-thioxo-thiazolidin-3-yl]-ethanesulfonic acid 4-fluoro-benzoylamide;

(Z) [5-(4-Hexyl-methyl-amino)benzylidene]-3-(5-oxo-4,5-dihydro-[1,2,4]oxadiazol-3-ylmethyl)-2-thioxo-thiazolidin-4one;

(Z) [5-(4-Propyl-piperidin-1-yl)-benzylidene]-3-(5-oxo-4,5-dihydro-[1,2,4]oxadiazol-3-ylmethyl)-2-thioxo-thiazolidin-4one;

(Z) [5-(4-Octahydro-isoquinolin-2-yl)-benzylidene]-3-(5-oxo-4,5-dihydro-[1,2,4]oxadiazol-3-ylmethyl)-2-thioxo-thiazolidin-4one;

(Z) 5-(4-Dipentylamino-benzylidene)-3-(5-oxo-4,5-dihydro-[1,2,4]oxadiazol-3-ylmethyl)-2-thioxo-thiazolidin-4-one; or

(Z) 5-(4-Dibutylamino-benzylidene)-3-(5-oxo-4,5-diydro-[1,2,4]oxadiazol-3-ylmethyl)-2-thioxo-thiazolidin-4-one.

Also provided is a pharmaceutical composition comprising a compound of Formula I together with a pharmaceutically acceptable diluent, excipient, or carrier therefor.

Also provided is a method of treating Alzheimer's disease, the method comprising administering to a patient having Alzheimer's disease a therapeutically effective amount of a compound of Formula I.

Also provided is a method of inhibiting the aggregation of amyloid proteins to form amyloid deposits, the method comprising administering to a patient in need of inhibition of the aggregation of amyloid proteins an amyloid protein aggregation inhibiting amount of a compound of Formula I.

Also provided is a method of imaging amyloid deposits, the method comprising the steps of:

a. introducing into a patient a detectable quantity of a labeled compound of Formula I;

b. allowing sufficient time for the labeled compound to become associated with amyloid deposits; and

c. detecting the labeled compound associated with the amyloid deposits.

In a preferred embodiment of the method of imaging, the patient has or is suspected to have Alzheimer's disease.

In another preferred embodiment of the method of imaging, the labeled compound is a radiolabeled compound.

In another preferred embodiment of the method of imaging, the labeled compound is detected using MRI.

### DETAILED DESCRIPTION OF THE INVENTION

The term “alkyl” means a straight or branched chain hydrocarbon Representative examples of alkyl groups are methyl, ethyl, propyl, isopropyl, isobutyl, butyl, tert-butyl, sec-butyl, pentyl, and hexyl.

Preferred alkyl groups are C₁-C₈ alkyl.

The term “alkoxy” means an alkyl group attached to an oxygen atom. Representative examples of alkoxy groups include methoxy, ethoxy, tert-butoxy, propoxy, and isobutoxy.

The term “halogen” includes chlorine, fluorine, bromine, and iodine.

The term “substituted” means that one or more hydrogen atom in a molecule has been replaced with another atom or group of atoms. For example, substituents include halogen, —OH, —CF₃, —NO₂, —NH₂, —NH(C₁-C₆alkyl), —N(C₁-C₆alkyl)₂, C₁-C₆ alkyl, —OC₁-C₆ alkyl, —CN, —CF₃, —CO₂H, and —CO₂C₁-C₆ alkyl.

The term “substituted phenyl” means a phenyl ring in which from 1 to 4 hydrogen atoms have been independently replaced with a substituent, preferably one selected from the list above. Typical “substituted phenyl” groups include 4-fluorophenyl, 3-chlorophenyl, 3-methyoxyphenyl, 4-trifiuoromethylphenyl, 4-dimethylamino phenyl, and 2,6-diuorophenyl.

The symbol “-” means a covalent bond.

The term pharmaceutically acceptable salt, ester, amide, and prodrug as used herein refers to those carboxylate salts, amino acid addition salts, esters, amides, and prodrugs of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palritate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci., 1977;66:1-19 which is incorporated herein by reference.)

Examples of pharmaceutically acceptable, nontoxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, nontoxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amides and C₁-C₂ dialkyl secondary amides are preferred. Amides of the compounds of the invention may be prepared according to conventional methods.

The term “prodrug” refers to compounds that are rapidly transformed in vivo to yield the parent compound of the above formulas, for example, by hydrolysis in blood. A thorough discussion is provided in T. Higuchi and V. Stella, Pro-Drugs as Novel Delivery Systems, Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The compounds of the present invention can exist in different stereoisometric forms by virtue of the presence of asymmetric centers in the compounds. It is contemplated that all stereoisometric forms of the compounds, as well as mixture thereof, including racemic mixtures, form part of this invention.

In the first step of the present method of imaging, a labeled compound of Formula I is introduced into a tissue or a patient in a detectable quantity. The compound is typically part of a pharmaceutical composition and is administered to the tissue or the patient by methods well-known to those skilled in the art, In the methods of the present invention, a compound can be administered either orally, rectally, parenterally (intravenous, by intramuscularly or subcutaneously), intracisternally, intravaginally, intraperitoneally, intravesicafly, locally (powders, ointments or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft- and hard-filled gelatin capsules using such excipients as lactose or milk sugar, as well as high molecular weight polyethyleneglycols, and the like.

Solid dosage forms such as tablets, dragees, capsules, pills, and granules can be prepared with coatings and shells, such as enteric coatings and others well known in the art They may contain opacifying agents, and can also be of such composition that they release the active compound or compounds in a certain part of the intestinal tract in a delayed manner. Examples of embedding compositions which can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, and sesame oil, glycerol, tetrahydrofurfuryl alcohol, polyethyleneglycols, and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable nonirritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers or propellants as may be required. Ophthalmic formulations, eye ointments, powders, and solutions are also contemplated as being within the scope of this invention.

In a preferred embodiment of the invention, the labeled compound is introduced into a patient in a detectable quantity and after sufficient time has passed for the compound to become associated with amyloid deposits, the labeled compound is detected noninvasively inside the patient. In another embodiment of the invention, a labeled compound of Formula I is introduced into a patient, sufficient time is allowed for the compound to become associated with amyloid deposits, and then a sample of tissue from the patient is removed and the labeled compound in the tissue is detected apart from the patient. In a third embodiment of the invention, a tissue sample is removed from a patient and a labeled compound of Formula I is introduced into the tissue sample. After a sufficient amount of time for the compound to become bound to amyloid deposits, the compound is detected.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or track the progress of Alzheimer's disease in a patient.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with amyloid deposits. The amount of time necessary can easily be determined by introducing a detectable amount of a labeled compound of Formula I into a patient and then detecting the labeled compound at various times after administration.

The term “associated” means a chemical interaction between the labeled compound and the amyloid deposit. Examples of associations include covalent bonds, ionic bonds, hydrophilic-hydrophilic interactions, hydrophobic-hydrophobic interactions, and complexes.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹¹C or ¹⁸F.

Another example of a suitable label in a compound of Formula I is an atom such as ¹³C, ¹⁵N, or ¹⁹F which can be detected using magnetic resonance imaging (MRI) which is also sometimes called nuclear magnetic resonance (NMR). In addition, the labeled compounds of Formula I may also be detected by MRI using paramagnetic contrast agents.

Another example of detection is electron paramagnetic resonance (EPR). In this case, EPR probes which are well-known in the art, such as nitroxides, can be used.

The imaging of amyloid deposits can also be carried out quantitatively so that the amount of amyloid deposits can be determined.

The present invention also provides a method of inhibiting the aggregation of amyloid proteins to form amyloid deposits, by administering to a patient in need of inhibition of the aggregation of amyloid protein an amyloid protein inhibiting amount of a compound of Formula I. Those skilled in the art are readily able to determine an amyloid inhibiting amount by simply administering a compound of Formula I to a patient in increasing amounts until the growth of amyloid deposits is decreased or stopped. The rate of growth can be assessed using imaging or by taking a tissue sample from a patient and observing the amyloid deposits therein.

A patient in need of inhibition of the aggregation of amyloid proteins is a patient having a disease or condition in which amyloid proteins aggregate. Examples of such diseases and conditions include Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Alzheimer's disease, Down's syndrome, Scrapie, Creutzfeldt-Jacob disease, Kura, Gerstmann-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, and Islets of Langerhans diabetes Type II insulinoma.

Also provided by the present invention are compounds of Formula I wherein one or more atom in the compound has been replaced with a radioisotope. The radioisotope can be any radioisotope. However, ³H, ¹²³I, ¹²⁵I, ¹³¹I, ¹³C, and ¹⁸F are preferred. Those skilled in the art are familiar with the procedure used to introduce a radioisotope into a compound. For example, compounds of Formula I wherein one carbon atom is ¹³C are readily prepared by standard method in organic chemistry.

The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well-known to those skilled in the art.

The examples presented below are intended to illustrate particular embodiments of the invention and are not intended to limit the scope of the specification, including the claims, in any manner.

### EXAMPLES

The compounds of the present invention can be generally prepared as illustrated in Scheme 1 below. With regard to Scheme 1, appropriately substituted amino benzaldehydes are commercially available or are prepared by reacting 4-fluorobenzaldehyde with an amine in the presence of a base such as potassium carbonate in a solvent such as dimethylacetaraide or dimethylformamide. N-substituted rhodanines that are not commercially available are prepared by condensing carbon disulfide and chloroacetic acid with the appropriate amine. The compounds of the present invention can be prepared by condensation of an appropriately N-substituted rhodanine with an appropriately substituted aromatic aldehyde in refluxing glacial acetic acid in the presence of sodium acetate. Other methods of preparing invention compounds will be readily available to those skilled in organic chemistry.

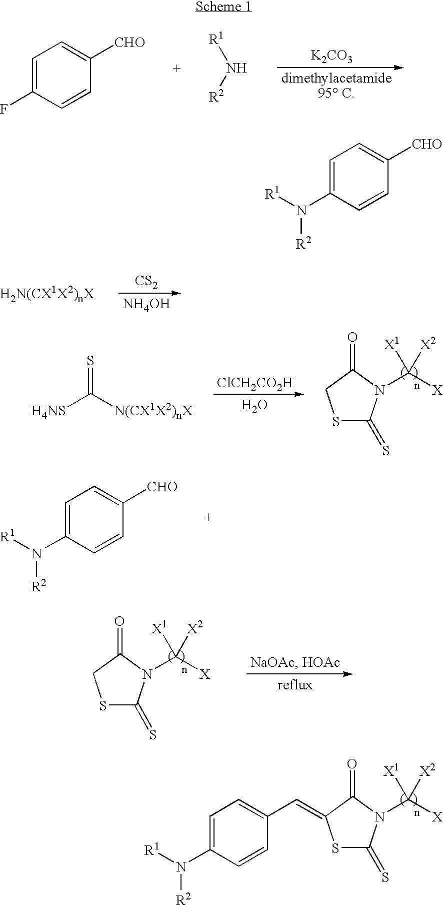

### Example 1

(Z) 2-{5[4-(Hexyl-methyl-amino)benzylidene] 4-oxo-2-thioxo-thiazolidin-3-yl}-ethanesulfonic acid

Step A:

Hexylmethyl amine (10 g, 86.8 mmol), 4-fluorobenzaldehyde (8.0 mL, 75.0 mmol) and potassium carbonate (12.4 g, 90.0 mmol) in dimethylacetamide (15 mL) are heated to 95° C. for 3 days with vigorous stirring. The reaction mixture is cooled, diluted with water (200 mL) and extracted with diethyl ether. The organic extract is dried (magnesium sulfate) and concentrated in vacuo. The resulting oil is purified by medium pressure liquid chromatography (MPLC) on silica gel eluting with 5% ethyl acetate/hexane to give 14.9 g of 4-(n-hexylmethylamino)benzaldehyde as a yellow oil.

Step B:

To a mixture of carbon disulfide (5.5 mL, 90 mmol) and ammonium hydroxide (20 mL) at 0° C. is added 2-aminoethane sulfonic acid (9.0 g, 72 mmol). The reaction mixture is warmed to room temperature and stirred for 18 hours then concentrated to dryness. This dithiocarbamate is added slowly to a cold (0° C.) solution of sodium chloroacetate (8.5 g, 75 mmol) in water (25 mL) made basic with sodium carbonate. The reaction mixture is warmed to room temperature and poured into a warm (70° C.) HCl solution (160 mL, 5 M) and heated to 90° C. for 1 hour. The reaction mixture is cooled, the product collected on a filter, washed with water, and dried to give 10.6 g of rhodanine-3-ethane sulfonic acid.

Step C:

4-(n-Hexylmethylamino)benzaldehyde (0.91 g, 4.14 mmol), rhodanine-3-ethyl sulfonic acid (1.00 g, 4.14 mmol), and sodium acetate acetate (0.42 g, 4.97 mmol) in acetic acid (15 mL) are heated to reflux for 15 hours with stirring. The reaction mixture is cooled, diluted with water, and the precipitated solids are collected by filtration, washed with water, and dried under vacuum to provide 0.85 g of the title compound as the sodium salt; melting point (mp)>250° C. Elemental analysis calculated for C₁₉H₂₅N₂O₄S₃Na(2.01 mol) H₂O: Calculated: C, 45.57; H, 5.84; N, 5.59. Found: C, 45.57; H, 5.62; N, 5.41.

### Example 2

(Z) 2-{5-[4-(Hexyl-methyl-amino)-benzylidene]-4-oxo-2-thioxo-thiazolidin-3-yl}-ethanesulfonic acid methylamide

(Z) 2-{5-[4-Hexyl-methyl-amino)-benzylidene]-4-oxo-2-thioxo-thiazolidin-3-yl}-ethanesulfonic acid (0.50 g, 1.13 mmol) is suspended in anhydrous (anh.) dichloromethane (25 mL) under N₂. To this mixture is added anh. dimethylformrnamide (6 mL) followed by oxalyl chloride (0.11 mL, 1.24 mmol). The reaction mixture is stirred for 3.5 hours at room temperature. Methyl amine (2.0 M in tetrahydrofuran (THF), 1.7 mL, 3.39 mmol) is added, and stirring is continued overnight. The reaction mixture is concentrated in vacuo and purified by MPLC (1% to 10% methanol (MeOH) in CH₂Cl₂) to give 0.106 g of the title compound, mp 147-149° C. Elemental analysis calculated for C₂₀H₂₉N₃O₃S₃: Calculated: C, 52.72; H, 6.42; N, 9.22. Found: C, 52.72; H, 6.38; N, 9.11.

### Example 3

(Z) 2-{5-[4-(Hexyl-methyl-amino)-benzylidene]-4-oxo-2-thioxo-thiazolidin-3-yl}-ethanesulfonic acid trifluoroacetyl-amide

(Z) 2-{5-[4-(Hexyl-methyl-amino)-benzylidene]4-oxo-2-thioxo-thiazolidin-3-yl}-ethanesulfonic acid (0.70 g, 1.58 mmol) is suspended in (anh.) dichloromethane (35 mL) under N₂. To this mixture was added anh. dimethylformamide (10 mL) followed by oxalyl chloride (3.6 mL, 7.27 mmol). The reaction mixture is stirred 15 hours at room temperature. In a separate flask, sodium hydride (60% dispersion on mineral oil, 0.76 g, 19.0 mmol) is suspended in anh. dimethylformamide (10 mL) under N₂. Triflouromethyl acetamide (2.15 g, 19.0 mmol) is slowly added. This mixture is stirred 20 minutes, added to the sulfonyl chloride solution, and stirred an additional 2 hours at room temperature. The reaction mixture is concentrated in vacuo and purified by MPLC (5% MeOH in CH₂Cl₂) to give 0.176 g of the title compound as an orange solid, mp 158-162° C. Elemental analysis calculated for C₂₁H₂₆F₃N₃O₄S₃: Calculated: C, 46.91; H, 4.87; N, 7.82. Found: C, 44.40; H, 4.59; N, 7.44.

### Example 4

(Z) 2-{5-[4(Hexyl-methyl-amino)-benzylidene]-4-oxo-2-thioxo-thiazolidin-3-yl}-N-methyl-acetamide

Ethyl chloroformate (0.23 mL, 2.42 mmol) is dissolved in anh. tetrahydrofuran (10 mL) and cooled to 0° C. under N₂. A solution of {5-[4-(hexylmethyl-amino)-benzylidene]-4-oxo-2-thioxo-thiazolidin-3-yl}-acetic acid (0.50 g, 1.27 mmol) and triethylamine (0.32 mL, 2.29 mmol) in anh. tetrahydrofuran (30 mL) is added dropwise. Stirred 2 hours at 0° C., then allowed to warm to room temperature. Methyl amine (2.0 M in THF, 1.91 mL, 3.81 mmol) is added and stirred at room temperature under N₂ for 15 hours. The reaction mixture is concentrated in vacuo and purified by MPLC (3% MeOH/CH₂Cl₂) to give 0.254 g of the title compound as an orange solid, mp 228-230° C. Elemental analysis calculated for C₂₀H₂₇N₃O₂S₂: Calculated: C, 59.23; H, 6.71; N, 10.36. Found: C, 58.82; H, 6.61; N, 10.00.

### Example 5

(Z) N-({5-[4-(Hexyl-methyl-amino)-benzylidene]4-oxo-2-thioxo-thiazolidin-3-yl}-acetyl)methanesulfonamide

A solution of {5-[4(hexyl-methyl-amino)-benzylidene]-4-oxo-2-thioxo-thiazolidin-3-yl}-acetic acid (0.50 g, 1.27 mmol) in anh. dimethylformamide (12 mL) is treated with 1,1′-carbonyldiimidazole (0.62 g, 3.82 mmol) and stirred at 60° C. for 4 hours under N₂. A solution of methylsulfonamide (0.36 g, 3.77 mmol) in anh. dimethylformamide (10 mL) is treated with sodium hydride (60% dispersion on mineral oil, 0.16 g, 4.04 mmol), stirred 4 hours under N₂, added to the reaction mixture and stirred for 15 hours at room temperature. The reaction mixture is poured into 1N HCl (100 mL) at 0° C. The resulting suspension is collected, washed with water and purified by MPLC (3% MeOH/CH₂Cl₂) to give 0.375 g of the title compound as an orange solid, mp 227-230° C. Elemental analysis calculated for C₂₀H₂₇N₃O₄S₃×0.28H₂O: Calculated: C, 50.61; H, 5.85; N, 8.85. Found: C, 50.37; H, 5.60; N, 8.62.

### Example 6

(Z) N-{5-[4-(Dipentylamino-benzylidene)₄-oxo-2-thioxo-thiazolidin-3-yl]-acetyl}-methanesulfonamide

Example 6 was prepared according to Example 5, except that [5-(4-dipentylamino-benzylidene)-4-oxo-2-thioxo-thiazolidin-3-yl]-acetic acid is substituted for {5-[4-(hexyl-methyl-amino)-benzylidene]-4-oxo-2-thioxo-thiazolidin-3-yl}-acetic acid, mp 134-138° C. Elemental analysis calculated for C₂₃H₃₃N₃O₄S₃×2.0CH₃OH: Calculated: C, 52.15; H, 7.18; N, 7.30. Found: C, 52.36; H, 7.05; N, 6.97.

### Example 7

(Z) C,C,C-Trifluoro-N-({5-[4-(hexyl-methyl-amino)-benzylidene]4-oxo-2-thioxo-thiazolidin-3-yl}-acetyl)methanesulfonamide

Example 7 was prepared according to Example 5, except that trifluoromethanesulfonamide is substituted for methanesulfonamide, mp 94-97° C. Elemental analysis calculated for C₂₀H₂₄F₃N₃O₄S₃×1.0C₆H₁₅N: Calculated: C, 49.98; H, 6.29; N, 8.97. Found: C, 50.01; H, 6.20; N, 8.77.

### Example 8

(Z) N-{5-[4-(Dipentylamino-benzylidene)-4-oxo-2-thioxo-thiazolidin-3-yl]-acetyl}-C,C,C-trifluoro-methanesulfonamide

Example 8 was prepared according to Example 6, except that trifluoromethanesulfonamide is substituted methanesulfonamide, mp 286-288° C. Elemental analysis calculated for C₂₃H₃₀F₃N₃O₄S₃: Calculated: C, 48.83; H, 5.35; N, 7.43. Found: C, 47.03; H, 4.90; N, 7.12.

### Example 9

(Z) N-({5-[4-(Hexyl-methyl-amino)benzylidene]-4-oxo-2-thioxo-thiazolidin-3-yl}-acetyl)benzenesulfonamide

Example 9 was prepared according to Example 5, except benzenesulfonamide is substituted for methanesulfonamide, mp 173-177° C. Elemental analysis calculated for C₂₅H₂₉N₃O₄S₃×0.33H₂O: Calculated: C, 55.85; H, 5.56; N, 7.82. Found: C, 55.81; H, 5.48; N, 7.59.

### Example 10

(Z) N-(2-{5-[4-(Hexyl-methyl-amino)-benzylidene]-4-oxo-2-thioxo-thiazolin-3-yl}-ethyl)-methanesulfonamide

Step A:

(Z) N-(2-{5-[4-(Hexyl-methyl-amino)-benzylidene]-4-oxo-2-thioxo-thiazolidin-3-yl}-ethyl)acetamide (0.50 g, 1.19 mmol) is suspended in 2N HCl (100 mL) and heated at reflux for 6 hours. The reaction mixture is cooled to room temperature, concentrated and purified by MPLC (5% MeOH/CH₂Cl₂) to give 0.380 g of (Z) N-(2-{5-[4-(hexyl-methyl-amino)-benzylidene]-4-oxo-2-thioxo-thiazolidin-3-yl}-ethyl)amine.

Step B:

Triethylamine (0.12 mL, 0.83 mmol) is added to (Z) N-(2-{5-[4-(hexyl-methyl-amino)-benzylidene]-4-oxo-2-thioxo-thiazolidin-3-yl}-ethyl)-amine (0.175 g, 0.46 mmol) in anh. dimethylformamide (20 mL), followed by methanesulfonyl chloride (0.07 mL, 0.93 mmol). The reaction is stirred overnight at room temperature under N₂, concentrated under vacuum, and purified by MPLC (1% MeOH/CH₂Cl₂) to give 50 mg of the title compound, mp 150-153° C. Elemental analysis calculated for C₂₀H₂₉N₃O₃S₃: Calculated: C, 52.72; H, 6.42; N, 9.22. Found: C, 52.84; H, 6.39; N, 9.14.

### Example 11

(Z) N(2-{5-[4-(Hexyl-methyl-amino)benzylidene]-4-oxo-2-thioxo-thiazolidin-3-yl}-ethyl)-benzenesulfonamide

Example 11 was prepared according to Example 10, except benzenesulfonyl chloride is substituted for methanesulfonyl chloride, mp 152-155° C. Elemental analysis calculated for C₂₅H₃₁N₃O₃S₃: Calculated: C, 58.00; H, 6.04; N, 8.12. Found: C, 58.28; H, 6.07; N, 8.05.

### Example 12

(Z) C,C,C-Trifluoro-N-(2-{5-[4-(hexyl-methyl-amino)benzyldene]-4-oxo-2-thioxo-thiazolidin-3-yl}-ethyl)methanesulfonamide

Example 12 was prepared according to Example 10, except trifluoromethanesulfonyl chloride is substituted for methanesulfonyl chloride, mp 170-173° C. Elemental analysis calculated for C₂₀H₂₆F₃N₃O₃S₃: Calculated: C, 47.14; H, 5.14; N, 8.25. Found: C, 47.43; H, 5.06; N, 8.16.

### Example 13

(Z) 2,2,2-Trifluoro-N-(2-{5-[4(hexyl-methyl-amino)benzylidene]-4-oxo-2-thioxo-thiazolidin-3-yl}-ethyl)-acetamide

Triflouroacetic anhydride (0.76 mL, 5.38 mmol) is added to (Z) N-(2-{5-[4-(hexyl-methyl-amino)-benzylidene]-4-oxo-2-thioxo-thiazolidin-3-yl}-ethyl)amine (0.48 g, 1.27 mmol) and sodium carbonate (0.57 g, 5.38 mmol) in anh. acetonitrile (30 mL). The reaction mixture is stirred overnight at room temperature under N₂, concentrated in vacuo, and purified by MPLC (5% MeOH/CH₂Cl₂) to give 0.100 g of the title compound. mp 136-139° C. Elemental analysis calculated for C₂₁H₂₆F₃N₃O₂S₂: Calculated: C, 53.26; H, 5.53; N, 8.87. Found: C, 53.28; H, 5.43; N, 8.79.

### Example 14

(Z) N-(2-{5-4-(Hexyl-methyl-amino)-benzylidene]-4-oxo-2-thioxo-thiazolidin-3-yl}-ethyl)-acetamide

Step A:

Rhodanine-3-ethyl acetamide was prepared according to Example 1, Step B, except acetyl ethylenediamine is substituted for 2-aminoethane sulfonic acid.

Step B:

Example 14 was prepared according to Example 1, Step C, except rhodanine-3-ethyl acetamide is substituted for rhodanine-3-ethyl sulfonic acid, mp 137-140° C. Elemental analysis calculated for C₂₁H_{29N}O₂S₂: Calculated: C, 60.11; H, 6.97; N, 10.01. Found: C, 60.38; H, 7.06; N, 10.01.

### Example 15

(Z) {5-[4-(n-Hexylmethylamine)-benzylidene]-4-oxo-2-thioxo-thiazolidin-3-yl}-methanesulfonic acid

By following the general procedure of Example 1, 4n-hexylmethylamino)benzaldehyde was reacted with rhodanine-3-methane sulfonic acid to give (Z) {5-[4-(n-hexylmethylamine)-benzylidene]-4-oxo-2-thioxo-thiazolidin-3-yl}-methanesulfonic acid.

MS 429 (M⁺).

### Example 16

(Z) 5-[4-(Hexyl-methyl-amino)benzylidene]-3-(1H-tetrazol-5-ylmethyl)-2-thioxo-thiazolidin-4-one

Step A:

Phthalimide (19.5 g, 0.132 mol) is suspended in anhydrous DMF (80 mL) under N₂. Potassium t-butoxide (17.8 g, 0.159 mol) is added slowly and the suspension stirred at room temperature for 10 minutes. Chloracetonitrile (10.1 mL, 0.159 mol) is then added and the mixture stirred overnight. Methanol (50 mL) is added and the mixture is concentrated in vacuo. The phthalimide acetonitrile is purified by MPLC (100% CH₂Cl₂) to give 16.0 g of white crystalline solid.

Step B:

Sodium azide (5.81 g, 89.4 mmol) and ammonium chloride (4.6 g, 85.9 mmol) are added to the phthalimide acetonitrile (16.0 g, 85.9 mmol) in DMF (100 mL) and heated to 100° C. for 6 hours. The solids are filtered and the filtrate concentrated in vacuo to give 17.25 g of the phthalimide methyl tetrazole as a white crystalline solid, mp 164-167° C.

Step C:

Hydrazine monohydrate (2.7 mL, 56.0 mmol) is added to the phthalimide methyl tetrazole (12.6 g, 55.0 mmol) suspended in EtOH (400 mL) and heated to reflux for 3 hours. The mixture is concentrated in vacuo and resuspended in 3N HCl (500 mL). The solids are filtered, washed with water, and dried to give 7.8 g of the tetrazole methyl amine as a white solid hydrochloride salt, mp 148-153° C.

Step D:

1H-tetrazol-5-ylmethyl-2-thioxo-thiazolidin-4-one was prepared from the tetrazole methyl amine as previously described in Example 1, Step B.

Step E:

5-[4-(Hexyl-methyl-amino)-benzylidene]-3(1H-tetrazol-5-ylmethyl)-2-thioxo-thiazolidin-4-one was prepared as previously described in Example 1, Step C, mp 198-200° C.

MS 417 (M⁺).

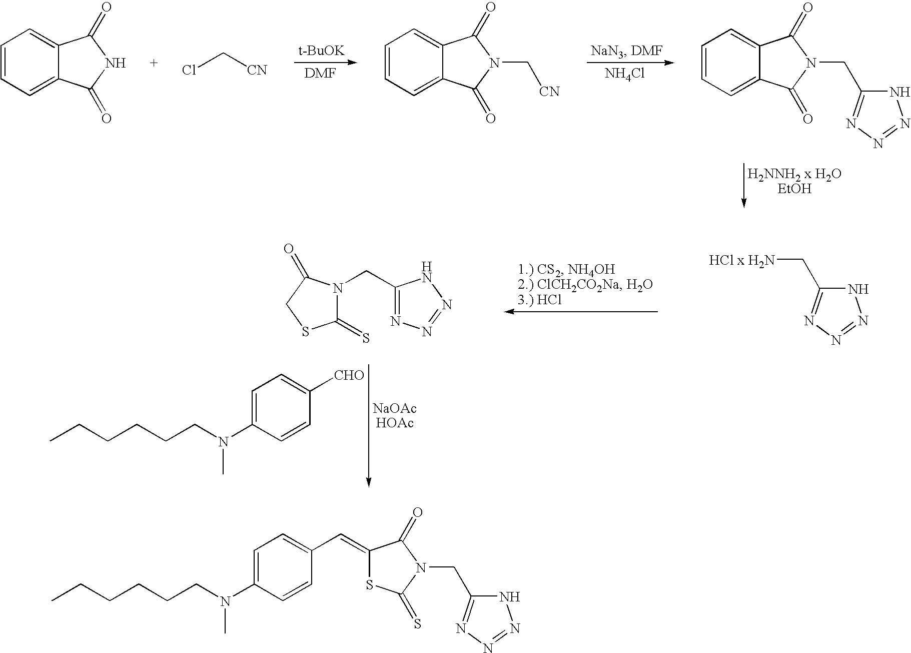

The following invention compounds were prepared by following the general procedures of the foregoing examples.

### Example 17

(Z) 5-(4-Dipentylamino-benzytidene)-3H-tetrazol-5-ylmethyl)-2-thioxo-thiazolidin-4-one. mp 208-210° C.

MS 459 (M+).

### Example 18

(Z) N-{[5-(4-Dibutylamino-benzylidene)-4-oxo-2-thioxo-thiazolidin-3-yl]-acetyl}-C,C,C-trifluoro-methanesulfonamide. mp 228-230° C.

MS 538 (M⁺).

### Example 19

(Z) N-{[5-(4-Dibutylamino-benzytidene)-4-oxo-2-thioxo-thiazolidin-3-yl]-acetyl}-benzenesulfonamide. mp 112-115° C.

MS 545 (M⁺).

### Example 20

(Z) 5-(4-Dibutylamino-benzylidene)-3(H-tetrazol-5-ylmethyl)-2-thioxo-thiazolidin-4-one. mp 243-246° C.

MS 431 (M⁺).

### Example 21

(Z) N-{2-[5-(4-Dibutylamino-benzylidene)-4-oxo-2-thioxo-thiazolidin-3-yl]-acetyl}-methanesulfonamide. mp 219-222° C.

MS 484 (M⁺).

### Example 22

(Z) N-{2-[5-(4-Dipentylamino-benzylidene)-4-oxo-2-thioxo-thiazolidin-3-yl]-acetyl}-benzenesulfonamide. mp 121-123° C.

MS 574 (M⁺).

### Example 23

(Z) 5-[(4aS,8aR)-4-(Octahydro-isoquinolin-2-yl)-benzylidene]-3-(1H-tetrazol-5-ylmethyl)-2-thioxo-thiazolidin-4-one. mp 247° C.

MS 441 (M⁺).

### Example 24

(Z) N-(2-{5-[(4aS,8aR)-4(Octahydro-isoquinolin-2-yl)benzylidene]-4-oxo-2-thioxo-thiazolidin-3-yl}-acetyl)-benzenesulfonamide. mp 222° C.

MS 556 (M⁺).

### Example 25

(Z) N-{2-[5-(4-Dibutylamino-benzylidene)-4-oxo-2-thioxo-thiazolidin-3-yl]-acetyl}-4-fluoro-benzenesulfonamide. mp 125° C.

MS 564 (M⁺).

### Example 26

(Z) 2-[5-(4-Dibutylamino-benzytidene)-4-oxo-2-thioxo-thiazolidin-3-yl]-ethanesulfonic acid 4-fluoro-benzoylamide. mp 191-192° C.

MS 578 (M⁺).

### Example 27

(Z) N-{2-[5-(4-Dipentylamino-benzylidene)-4-oxo-2-thioxo-thiazolidin-3-yl]-acetyl}-4-fluoro-benzenesulfonamide. mp 130-132° C.

MS 592 (M⁺).

### Example 28

(Z) 2-[5-(4-Dibutylamino-benzylidene)-4-oxo-2-thioxo-thiazolidin-3-yl]-ethanesulfonic acid benzoylamide. mp 183° C.

MS 560 (M⁺).

### Example 29

(Z) 2-{5-[4-(Octahydro-isoquinolin-2-yl)-benzylidene]-4-oxo-2-thioxo-thiazolidin-3-yl}-ethanesulfonic acid benzoylamide. mp 213° C.

MS 570 (M⁺).

### Example 30

(Z) 2-{5-[4(Octahydro-isoquinolin-2-yl)benzylidene]-4-oxo-2′-thioxo-thiazolidin-3-yl}-ethanesulfonic acid 4-fluoro-benzoylamide. mp 248° C.

MS 588 (M⁺).

### Example 31

(Z) 2-[5-(4-Dipentylamino-benzylidene)-4-oxo-2-thioxo-thiazolidin-3-yl]-ethanesulfonic acid 4-fluoro-benzoylamide. mp 195-196° C.

MS 604 (M⁻).

### Example 32

(Z) 3-(5-Hydroxy-4-oxo-4H-pyran-2-ylmethyl)-5-[4-(octahydro-isoquinolin-2-yl)benzylidene]-2-thioxo-thiazolidin-4-one

Step A:

Chlorokojic acid (5.0 g, 31.1 mmol) and sodium azide (2.06 g, 31.8 mmol) are stirred in DMF (20 mL) overnight at room temperature. The reaction mixture is diluted with water (120 mL) and the white precipitation is collected, washed with water, and dried to give 3.72 g of the azide as a white solid.

Step B:

Triphenylphosphine (3.53 g, 13.45 mmol) is slowly added to the kojic acid azide (1.5 g, 8.97 mmol) in THF (20 mL). The evolution of gas is immediate. Water (0.8 mL) is added and the reaction heated to 55° C. for 18 hours. The reaction mixture is cooled and solids are collected and washed with diethyl ether. The kojic acid amine (0.74 g) is obtained as a light tan solid.

Step C:

The 2-thioxo-thiazolidin-4-one of kojic acid is prepared as previously described in Example 1.

Step D:

3(5-Hydroxy-4-oxo-4H-pyran-2-ylmethyl)-5-[4-(octahydro-isoquinolin-2-yl)-benzylidene]-2-thioxo-thiazolidin-4-one is prepared as previously described in Example 1, Step C, mp 238.

MS 483 (M⁺)

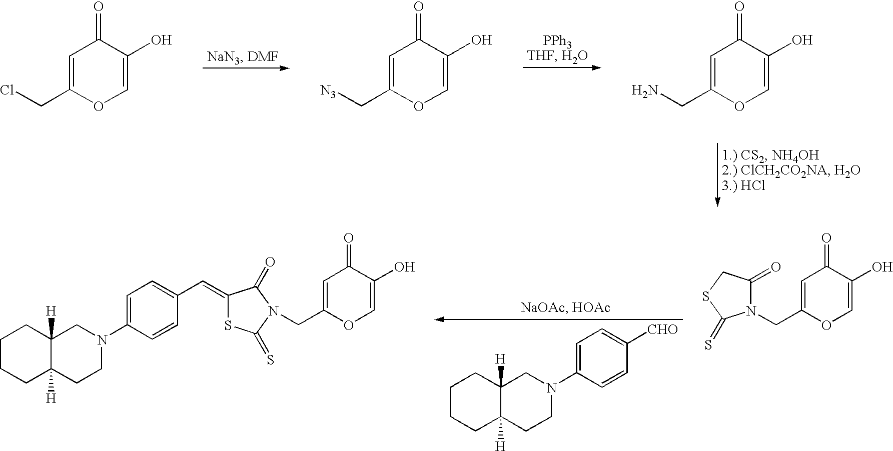

The following compounds of Formula I (Examples 33-62) were prepared according to the general procedures described above.

### Example 33

(Z) 5-(4-Dibutylamino-benzylidene)-3-(5-hydroxy-4-oxo-4H-pyran-2-ylmethyl)-2-thioxo-thiazolidin-4-one. mp 226-227° C.

MS 473 (M⁺).

### Example 34

(Z) 3-(5-Hydroxy-4-oxo-4H-pyran-2-ylmethyl)-5-[4-(4-propyl-piperidin-1-yl)-benzylidene]-2-thioxo-thiazolidin-4-one. mp 253° C.

MS 471 (M⁺).

### Example 35

(Z) 5-[4[(4-Propyl-piperidin-1-yl)-benzylidene]-3-(1H-tetrazol-5-ylmethyl)-2-thioxo-thiazolidin-4-one. mp 263° C.

MS 429 (M⁺).

### Example 36

(Z) N-(2-{4-Oxo-5-[4-(4-propyl-piperidin-1-yl)-benzylidene]-2-thioxo-thiazolidin-3-yl}-acetyl)benzenesulfonamide. mp 201° C.

MS 544 (M⁺).

### Example 37

(Z) N-(2-{4-Oxo-5-[4-(4-propyl-piperidin-1-yl)-benzylidene]-2-thioxo-thiazolidin-3-yl}-acetyl)methanesulfonamide. mp 254° C.

MS 482 (M⁺).

### Example 38

(Z) 4-Fluoro-N-(2-{5-[(4aS,8aR)-4-(octahydro-isoquinolin-2-yl)-benzylidene]-4-oxo-2-thioxo-thiazolidin-3-yl}-acetyl)-benzenesulfonamide. mp 220-221° C.

MS 574 (M⁺).

### Example 39

(Z) 4-Fluoro-N-(2-{4-oxo-5-[4-(4-propyl-piperidin-1-yl)-benzylidene]-2-thioxo-thiazolidin-3-yl}-acetyl)-benzenesulfonamide. mp 197° C.

MS 562 (M⁺).

### Example 40

(Z) 2-[5-(4-Hexyl-methyl-amino-benzylidene)-4-oxo-2-thioxo-thiazolidin-3-yl]-ethanesulfonic acid 4-fluoro-benzoylamide.

MS 550 (M⁺).

### Example 41

(Z) N-({5-[4[(Octahydro-isoquinolin-2-yl)-benzylidene]-4-oxo-2-thioxo-thiazolidin-3-yl}-acetyl)methanesulfonamide

### Example 42

(Z) N-({5-[4[(Octahydro-isoquinolin-2-yl)-benzylidene]-4-oxo-2-thioxo-thiazolidin-3-yl}-acetyl)-C,C,C-trifluoro-methanesulfonamide

### Example 43

(Z) N-(2-{4-Oxo-5-[4-(4-propyl-piperidin-1-yl)-benzylidene]-2-thioxo-thiazolidin-3-yl}-acetyl)-C,C,C-trifluoro-methanesulfonamide

### Example 44

(Z) 2-[5-(4-Dibutylamino-benzylidene)-4-oxo-2-thioxo-thiazolidin-3-yl]-ethanesulfonic acid methylamide

### Example 45

(Z) 2-[5-(4-Dipentylamino-benzylidene)-4-oxo-2-thioxo-thiazolidin-3-yl]-ethanesulfonic acid methylamide

### Example 46

(Z) 2-[5-(4-Hexyl-methyl-amino-benzylidene)-4-oxo-2-thioxo-thiazolidin-3-yl]-ethanesulfonic acid methylamide

### Example 47

(Z) N-2-{4-Oxo-5-[4-(4-propyl-piperidin-1-yl)benzylidene]-2-thioxo-thiazolidin-3-yl}-ethanesulfonic acid methylamide

### Example 48

(Z) 2-{5-[4-(Octahydro-isoquinolin-2-yl)benzylidene]-4-oxo-2-thioxo-thiazolidin-3-yl}S-ethanesulfonic acid methylamide

### Example 49

(Z) 2-{5-[4-(Octahydro-isoquinolin-2-yl)-benzylidene]-4-oxo-2-thioxo-thiazolidin-3-yl}S-ethanesulfonic acid trifluoroacetylamide

### Example 50

(Z) N-2-{4-Oxo-5-[4-(4-propyl-piperidin-1-yl)benzylidene]-2-thioxo-thiazolidin-3-yl}-ethanesulfonic acid trifluoroacetylamide

### Example 51

(Z) 2-[5-(4-Dipentylamino-benzylidene)-4-oxo-2-thioxo-thiazolidin-3-yl]-ethanesulfonic acid trifluoroacetylamide

### Example 52

(Z) 2-[5-(4-Dibutylamino-benzylidene)-4-oxo-2-thioxo-thiazolidin-3-yl]-ethanesulfonic acid trifluoroacetylamide

### Example 53

(Z) 2-[5-(4-Dipentylamino-benzylidene)-4-oxo-2-thioxo-thiazolidin-3-yl]-ethanesulfonic acid benzoylamide

### Example 54

(Z) 2-[5-(4-Hexyl-methyl-benzylidene)-4-oxo-2-thioxo-thiazolidin-3-yl]-ethanesulfonic acid benzoylamide

### Example 55

(Z) N-2-{4-Oxo-5-[4-(4-propyl-piperidin-1-yl)benzylidene]-2-thioxo-thiazolidin-3-yl}-ethanesulfonic acid benzoylamide

### Example 56

(Z) N-2-{4-Oxo-5-[4-(4-propyl-piperidin-1-yl)-benzylidene]-2-thioxo-thiazolidin-3-yl}-ethanesulfonic acid 4-fluoro-benzoylamide

### Example 57

(Z) 2-[5-(4-Hexyl-methyl-benzylidene)-4-oxo-2-thioxo-thiazolidin-3-yl]-ethanesulfonic acid 4-fluoro-benzoylamide

### Example 58

(Z) [5-(4-Hexyl-methyl-amino)-benzylidene]-3-(5-oxo-4,5-dihydro-[1,2,4]oxadiazol-3-ylmethyl)-2-thioxo-thiazolidin-4-one

### Example 59

(Z) [5-(4-Propyl-piperidin-1-yl)-benzylidene]-3-(5-oxo-4 dihydro-[1,2,4]oxadiazol-3-ylmethyl)-2-thioxo-thiazolidin-4-one

### Example 60

(Z) [5-(4-Octahydro-isoquinolin-2-yl)benzylidene]-3-(5-oxo-4,5-dihydro-[1,2,4]oxadiazol-3-ylmethyl)-2-thioxo-thiazolidin-4-one

### Example 61

(Z) 5-(4-Dipentylamino-benzylidene)-3-(5-oxo-4,5-dihydro-[1,2,4]oxadiazol-3-ylmethyl)-2-thioxo-thiazolidin-4-one

### Example 62

(Z) 5-(4-Dibutylamino-benzylidene)-3-(5-oxo-4,5-dihydro-[1,2,4]oxadiazol-3-ylmethyl)-2-thioxo-thiazolidin-4-one

Other typical invention compounds that can be prepared by following the foregoing general methods include:

(Z) N-(2-{5-[(4-Hexylmethylamino)-naphthalan-1-ylmethylene]-4-oxo-2-thioxo-thiazolidin-3-yl}-acetyl)benzenesulfonamide;

(Z) 2-[5-(1-Ethyl-2,3 dihydro-1H-indol-5-ylmethylene)4-oxo-2-thioxo-thiazolidin-3-yl]-ethanesulfonic acid;

(Z) 3-[5-(1-Isopropyl-1,2,3,4-tetrahydro-quinolin-6-yl-methylene)-4-oxo-2-thioxo-thiazolidin-3-yl]-propanesulfonic acid; and

(Z) 1-[5-(1-tert.Butyl-1,2,3,4-tetrahydro-quinolin-5-yl-methylene)-4-oxo-2-thioxo-thiazolidin-3-yl]-1-methylethanesulfonic acid.

### BIOLOGICAL EXAMPLES

Representative compounds of Formula I have been evaluated in the following standard in vitro and in vivo assays which are commonly used to indicate clinical utility in inhibiting amyloid formation and to treat diseases associated with amyloid, such as Alzheimer's disease.

Amyloid Assays

BASSR (Beta-Amyloid Self-Seeding Radioassay)

An assay for inhibitors of self-seeded amyloid fibril growth

Materials:

Stock Solutions:

Assay Buffer—50 mM sodium phosphate, pH 7.5, 100 mM NaCl, 0.02% NaN₃, 1 M urea (filter and store at 4° C.)

Soluble Aβ(1-40) peptide (Bachem, Torrance, Calif.)—2.2 mg/mL in deionized H₂O (stored in aliquots at −20° C., keep on ice when thawed) will self-seed after 1 week storage. Typically, the solution should be stored until no lag phase is seen in the assay.

¹²⁵I-labeled Aβ(1-40)—150K-350K cpm/μL in 100% acetonitrile—0.1% trifluoroacetic acid (TFA)—1% β-mercaptoethanol (aliquots stored at −20° C.). ¹²⁵I-labeled Aβ(1-40) can be made in accordance with the procedure set forth by H. Levine, III in Neurobiol. Aging, 16:755 (1995), which is hereby incorporated by reference, or this reagent may be purchased from Amersham, Arlington Heights, Ill.

Final assay conditions: 30 μM soluble Aβ(1-40) in deionized water in assay buffer+20-50K cpm ¹²⁵I-labeled Aβ(1-40) per assay. Compound to be tested is dissolved in dimethylsulfoxide (DMSO), typically 5-50 mM stock, such that the final concentration of DMSO is <1% v/v in the assay.

Assay: Reaction mixture for 50 assays (on ice) is comprised of 0.1-0.2 μL of ¹²⁵I-labeled A ¹²⁵I-labeled Aβ(1-40)+1 μL of soluble Aβ(1-40)+13.5 μL assay buffer per assay. The following are the amounts of the components of the reaction mixture sufficient for 50 assay wells.

5-10 mL ¹²⁵I-labeled Aβ(140) dried down

675 μL assay buffer

50 μL soluble Aβ(140)

Assay Method

1) Prepare reaction mixture above by mixing components and storing on ice.

2) Pipet 14.5 μL of reaction mixture into each of 50 wells on a polypropylene U-bottom 96-well microtiter plate on ice (Costar 3794).

3) Add 1.7 μL of diluted compound to be tested to each well in a column of eight, including solvent control. Serial 3-fold dilutions from 1 mM (100 μM final) in assay buffer−urea=7 dilutions+zero. Each 96-well plate can therefore accommodate 11 samples+1 Congo Red control (0.039-5 μM final in 2-fold steps).

4) Seal the plate with aluminum film (Beckman 538619) and incubate for 10 minutes on ice.

5) Raise the temperature to 37° C. and incubate for 3 to 5 hours (depending on the lot of the peptide).

6) Remove the aluminum film and add 200 μL/well of ice cold assay buffer with urea, collecting the radiolabeled fibrils by vacuum filtration through 0.2 μm pore size GVWP filters in 96-well plates (Millipore MAGV N22, Bedford, Mass.). Determine the radioactivity of the filters using standard methods well-known to those skilled in the art.

BASST (Beta-Amyloid Self-Seeding, ThioflavinT)

An assay for inhibitors of self-seeded amyloid fibril growth

Methods:

Materials

Stock Solutions:

Assay Buffer—50 mM sodium phosphate, pH 7.5, 100 mM NaCl, 0.02% NaN₃, 1 M urea (filter and store at 4° C.).

Soluble Aβ(1-40)—2.2 mg/mL in deionized H₂O (store in aliquots at −20° C., keep on ice when thawed) will self-seed after 1 week storage. Typically, the solution should be stored until no lag phase is seen in the assay.

Final assay conditions: 30 μM soluble Aβ(1-40) in deionized water in assay buffer. Compound to be tested is dissolved in DMSO, typically 5-50 mM stock, such that the final concentration of DMSO is <1% v/v in the assay.

Assay: Reaction mixture for 50 assays (on ice) comprised of 1 μL of soluble Aβ(1-40)+13.5 μL assay buffer per assay. The following are the amounts of the components of the reaction mixture that result in each of the 50 assay wells.

50 μL soluble Aβ(1-40)

675 μL assay buffer

Assay Method

1) Prepare the reaction mix above by mixing the components and storing on ice.

2) Pipet 14.5 mL of reaction mixture into each of 50 wells of a polystyrene U-bottom 96-well microtiter plate (Corning 25881-96) on ice.

3) Add 1.7 mL of diluted compound to be tested to each well in a column of eight, including solvent control. Serial 3-fold dilutions from 1 mM (100 μM final) in assay buffer−urea=7 dilutions+zero. Each 96-well plate can therefore accommodate 11 samples+1 Congo Red control (0.039-5 μM final in 2-fold steps).

4) Seal the plate with aluminum film and incubate for 10 minutes on ice.

5) Raise the temperature to 37° C. and incubate for 3 to 5 hours (depends on the lot of the peptide).

6) Remove the aluminum film and add 250 μL/well of 5 μM thioflavin T (ThT) [T-3516, Sigma-Aldrich] in 50 mM glycine-NaOH, pH 8.5. Read fluorescence on a plate reader (ex=440 nm/20 nm; em=485 nm/20 nm) within 5 minutes.

BAPA (Beta-Amyloid Peptide Aggregation)

This assay is used to provide a measure of inhibition by a compound against the aggregation behavior of the beta amyloid peptide.

The purpose of this assay is to provide a higher volume method of assaying the amount of beta amyloid aggregation using an endpoint assay based on filtration. In this assay, hexafluoroisopropanol (HFIP) is used to break down the initial amyloid peptide to a monomer state and use a concentration of 33 μM which is high enough so that aggregation will occur at pH 6.0 in several hours.

Methods:

β-Amyloid Peptide Aggregation, pH 6.0 (BAPA)

In a 96-well plate (Costar 3794), we add 25 mL 50 mM Phosphate Buffer, pH 6.0, 10 μL 0.5 mg/mL AP (140) peptide in 20% HFIP+0.1 μL/assay radioiodinated ¹²⁵I Aβ(1-40) [¹²⁵I Aβ(1-40)], and 1 μL of the compound to be tested starting at 50 mM with a concentration of DMSO <1%. Then, we incubate for 2 to 4 hours at room temperature. We stop the reaction with 200 mL of 50 mM phosphate buffer, pH 6.0, and filter it through a 0.2 μm 96-well filter plate (Millipore MAGU N22). We wash the filter plate with 100 μL of the same phosphate buffer. Aggregation was detected on a Microbeta counter after impregnating the filters with Meltilex (1450-441) and is corrected for background.

BATYM Assay

Methods:

Required Aβ(1-42)(California Peptide) was dried from its hexafluoroisopropanol (HFIP) stock solution. The Aβ(1-42) was dissolved in dimethylsulfoxide (DMSO) and then mixed with phosphate buffered saline, (PBS) (pH 7.4). The mixed Aβ(1-42) solution was filtered with a 0.2 μm Omnipore membrane syringe filter (Millipore, Bedford, Mass.). The compound to be tested in DMSO (50 times concentrate) was put into each well (0.5 μL/well) of a 96-well plate. The Aβ(1-42) solution was added into each well (24.5 μL/well). The plate was centrifuged at 1,000 g for 5 minutes and incubated at 37° C. for 1 day (Aβ 1-42; final concentration 100 μM).

After incubation Thioflavin T (ThT) (30 μM) solution in glycine-NaOH buffer (pH 8.5, 50 mM) was added into each well (250 μL/well), fluorescence was measured (ex. 440/20 nm; em 485/20 nm) using a fluorescence plate reader. The inhibitory activity was calculated as the reduction of fluorescence with the following formula:

inhibition (%)={(F(Aβ)−F(Aβ+compound)}/{F(Aβ)−F(solvent+compound)}×100

The IC₅₀s were calculated by a curve fitting program using the following equation. The data were obtained from two different experiments in triplicate.

Inhibition(x)=100−100/{1+(x/IC₅₀)ₙ},

x=concentration of tested compound (M),

IC₅₀=(M),

n=Hill coefficient.

Representative compounds of Formula I have exhibited inhibitory activities (IC₅₀) ranging from about 0.1 μm to greater than 100 μm in the foregoing assays. The results of these assays for several specific compounds of the present invention are shown in Table 1 below.

TABLE 1
Amyloid Inhibitory Activity

| Example | BASSR | BASST | BATYM | BAPA |
|---|---|---|---|---|
| No. | IC₅₀ μM | IC₅₀ μM | IC₅₀ μM | IC₅₀ μM |
| 1 | >100 | 1 | 1.72 | 23 |
| 2 | >100 | 0.8 | 2.32 | 7 |
| 3 | 8 | 0.3 | 2.24 | >100 |
| 4 | >100 | 3 | 8.26 | 6 |
| 5 | 9 | 0.8 | 1.83 | >10 |
| 6 | >100 | 28.5 | 2.92 | 3 |
| 7 | 1 | 0.4 | 1.7 | 70 |
| 8 | >100 | 1 | 1.96 | 2.5 |
| 9 | >100 | 1.75 | 2.15 | 4 |
| 10 | >100 | 1.1 | 2.51 | 8 |
| 11 | >100 | 9 | 2.19 | 25 |
| 12 | >100 | 1 | 1.64 | 26 |
| 13 | >100 | 10 | 3.5 | 28 |
| 14 | >100 | 0.22 | >100 | 30 |
| 15 | >100 | >100 | 3.39 | 8 |
| 16 | 7 | 3 | 1.94 | 38 |
| 17 | 10, 5 | 1, 1.5 | 1.87 | 2 |
| 18 | 5 | 0.6 | 1.88 | 122 |
| 19 | 5 | 1.1 | 1.77 | 129 |
| 20 | 1, 8.5 | 2 | 2.04 | >100 |
| 21 | >100 | | 2.91 | >100 |
| 22 | 3 | 1 | 1.99 | >60 |
| 23 | >100 | 1, 0.5 | 3.33 | 30 |
| 24 | 6.5, 10, 6 | 1, 0.8 | 3.84 | 60 |
| 25 | 6 | 0.6, 0.5 | 2.38 | 100 |
| 26 | 6.1, 10 | 0.5, 10 | 2.14 | |
| 27 | 7 | 2 | 2.02 | 93 |
| 28 | 21 | 0.3 | 2.19 | 69 |
| 29 | 11 | 0.6 | 3.42 | >100 |
| 30 | >100 | 0.3 | 2.26 | >60, >60 |
| 31 | 9 | 0.3 | 3.11 | >100 |
| 32 | >100 | 1 | 2.55 | 46 |
| 33 | >100 | 2 | 3.03 | >60 |
| 34 | >100 | 2 | 2.38 | >60 |
| 35 | 15 | 0.4 | 3.31 | >60 |
| 36 | >100 | 1 | 3.3 | >60 |
| 37 | 50 | 0.4 | 3.83 | >60 |
| 38 | 10, 5 | 0.7, 0.1 | 2.54 | 66 |
| 39 | >100, 21 | 1, 0.1 | 2.86 | 37 |
| 40 | | | 2.84 | |

Activity of the invention compounds is also evaluated in standard in vivo assays commonly used to evaluate agents to treat diseases related to aggregation of amyloid proteins, especially Alzheimer's disease. Such assays are described by Axelrad et al., Lab. Invest., 1982;47(2):139-146; and by Stenstad et al., J. Biochem., 1994;303(Pt 2):663-670. In one assay, amyloid protein is induced into the spleen of mice by subcutaneous injections of silver nitrate, Freund's complete adjuvant, and an intravenous injection of amyloid enhancing factor. Silver nitrate is administered each day through Day 11. Test compounds are administered to the mice daily starting on Day 1 through Day 11. On Day 12, the animals are sacrificed, and the spleens are removed, histologically prepared, stained with Congo red, and the percent area of the spleen occupied by birefringent, Congo red-stained amyloid is quantitated microscopically.

Another in vivo assay in which the invention compounds are evaluated uses transgenic mice. The mice bear a human β-amyloid precursor protein transgene with a prior promoter and are described by Hsiao et al., “Correlative Memory Deficits, Aβ Elevation, and Amyloid Plaques in Transgenic Mice,” Science 1996;274:99-102. These transgenic mice develop β-amyloid deposits at about 9 months of age. By 15 months, diffuse and compact senile plaques are abundant, primarily in the neocortex, olfactory bulb, and hippocampus. Invention compounds are administered orally to the mice beginning at the age of 8 months (just prior to the onset of amyloid deposits) and continuing for several months (up to about age 14-18 months). The animals are then sacrificed, and the brains are removed. The amount of amyloid in the brain is quantitated both histologically and biochemically.

The above data establishes that representative invention compounds are active in standard assays used to measure inhibition of protein aggregation. The compounds are thus useful to clinically inhibit amyloid protein aggregation and to image amyloid deposits for diagnostic use. The compounds will be used in the form of pharmaceutical formulations, and the following examples illustrate typical compositions.

EXAMPLE 63
Tablet Formulation

| Ingredient | Amount |
|---|---|
| Compound of Example 1 | 50 mg |
| Lactose | 80 mg |
| Cornstarch (for mix) | 10 mg |
| Cornstarch (for paste) | 8 mg |
| Magnesium Stearate (1%) | 2 mg |
| | 150 mg |

The compound of Example 1 is mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium sterate and compressed into a tablet. The tablets are administered to a patient at the rate of 1 to 4 each day for prevention of amyloid protein aggregation and treatment of Alzheimer's disease.

### EXAMPLE 64

### Parenteral Solution

In a solution of 700 mL of propylene glycol and 200 mL of water for injection is added 20.0 g of Compound No. 26) (from Example 26). The mixture is stirred and the pH is adjusted to 5.5 with hydrochloric acid. The volume is adjusted to 1000 mL with water for injection. The solution is sterilized, filled into 5.0 mL ampoules, each containing 2.0 mL (40 mg of Compound No. 26), and sealed under nitrogen. The solution is administered by injection to a patient suffering from medullary carcinoma of the thyroid and in need of treatment.

### EXAMPLE 65

### Patch Formulation

Ten milligrams of 5-[4-(4-propyl-piperidin-1-yl)-benzylidene]-3-(1H-tetrazol-5-yl-methyl)-2-thioxo-thiazolidin-4-one is mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of amyloid polyneuropathy.

The invention and the manner and process of making and using it, are now described in such full, clear, concise; and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. (Cancelled).

2. A method in accordance with claim 8 wherein R¹ is methyl, and R² is pentyl or hexyl.

3. A method in accordance with claim 8 wherein the 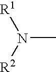
group is located at the para position on the phenyl ring.

4.-7. (Cancelled).

8. A method of imaging amyloid deposits, the method comprising the steps of:
a. introducing into a patient a quantity of a labeled compound having the Formula I: 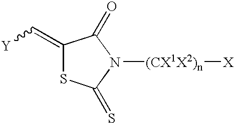
or a pharmaceutically acceptable salts thereof, wherein: 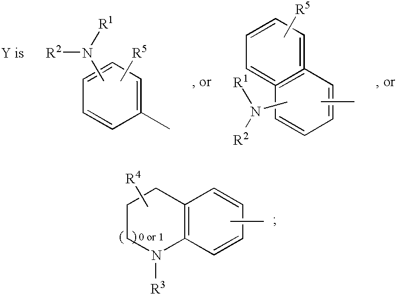
each n is independently 1 to 3 inclusive: X¹ and X² are independently hydrogen or C₁-C₈ alkyl, or —(CH₂)_{y}-Z: v is 0 to 4 inclusive: Z is hydrogen, C alkyl, C₁-C₈ perfluoroalkyl, C₂-C₈ alkenyl, phenyl substituted phenyl naphthyl, substituted naphthyl, —OH, —OC₁-C₈ alkyl, —SC₁-C_alkyl, —SO₃H, —CO₂H, —CO₂C₁-C₈ alkyl, 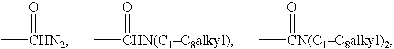
—NH₂, —NH(C₁-C₈alkyl), —N(C₁-C₈alkyl)₂, 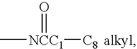
guanidinyl, thienyl, imidazolyl thiazolyl or indolyl: R¹ and R² are independently C₁-C₈alkyl or —(CH₂)ₙ—C₃-C₆cycycloalkyl, —(CH₂)ₙ-phenyl, or R¹ and R² taken together with the nitrogen atom to which they are attached form a cyclic structure selected from the group consisting of 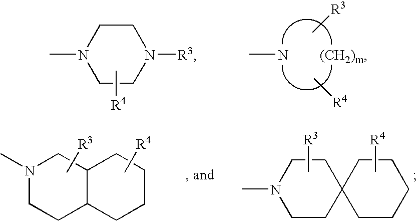
where R³ and R⁴ independently are hydrogen. C₁-C₈ alkyl, —(CH₂)ₙ-phenyl, or —(CH₂)ₙ cycloalkyl; R⁵ is hydrogen, C₁-C₈ alkyl, halogen, or —CF₃; each m is 2 to 8 inclusive: 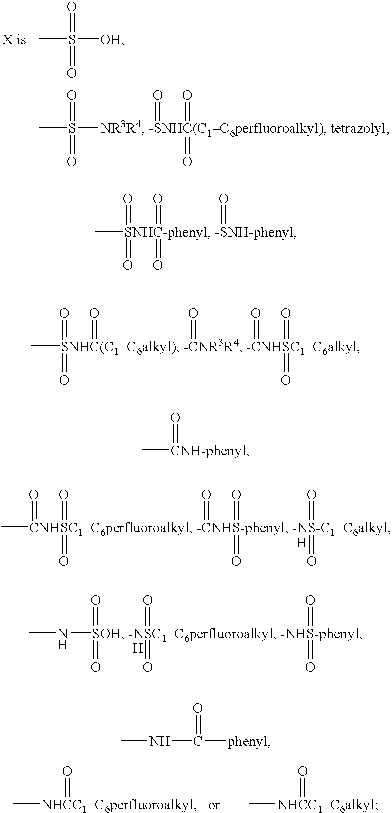
wherein phenyl includes substituted phenyl;
b. allowing sufficient time for the labeled compound to become associated with deposits; and
c. detecting the labeled compound associated with the amyloid deposits.

9. The method of claim 8 wherein the patient has or is suspected to have Alzheimer's disease.

10. The method of claim 8 wherein the labeled compound is a radiolabeled compound.

11. The method of claim 8 wherein the labeled compound is detected using MRI.
